(19) United States
(12) Patent Application Publication
(10) Pub. No.: US 2022/0036970 A1
(43) Pub. Date: Feb. 3, 2022

(54) METHODS AND SYSTEMS FOR DETERMINATION OF GENE SIMILARITY

(71) Applicant: Regeneron Pharmaceuticals, Inc., Tarrytown, NY (US)

(72) Inventors: Biming Wu, Tarrytown, NY (US); Suganthi Balasubramanian, Tarrytown, NY (US); Jeffrey Reid, Tarrytown, NY (US)

(21) Appl. No.: 17/345,477

(22) Filed: Jun. 11, 2021

Related U.S. Application Data
(60) Provisional application No. 63/038,504, filed on Jun. 12, 2020.

(51) Int. Cl. G16B 20/00

(52) U.S. Cl. CPC G16B 20/00

(57) ABSTRACT

Methods are disclosed for determining similarities between genes.

### CROSS REFERENCE TO RELATED PATENT APPLICATION

This application claims priority to U.S. Provisional Application No. 63/038,504, filed Jun. 12, 2020, herein incorporated by reference in its entirety.

### BACKGROUND

The application of high-throughput DNA sequencing to human cohorts has enabled genetic discovery, from the development of comprehensive catalogs of rare and common genetic variations (Genomes Project, C., et al., Nature 2010; 467: 1061; Tennessen J A, et al., Science 2012; 337: 64) to the elucidation of novel causal genes in Mendelian diseases (Chong J X, et al., Am J Hum Genet 2015; 97: 199; Yang Y, et al., JAMA, 2014; 312:1870), and rare variants have been implicated in common complex diseases (Do R, et al., Nature 2015; 518: 102; Holm H, et al., Nat Genet 2011; 43: 316; Steinberg S, et al., Nat Genet, 2015; 47: 445).

Recent discoveries have been aided by discovery of rare “human knockouts (MacArthur D G, et al., Science 2012; 335:823; Sulem P, et al., Nat Genet 2015; 47: 448; Lim E T, et al., PLoS Genet 2014; 10: e1004494). In some cases, sequence databases are linked to epidemiological data (Li A H, et al., Nat Genet 2015; 47: 640) or clinical phenotypes captured in structured clinical records (Sulem P, et al., Nat Genet 2015; 47: 448; Lim E T, et al., PLoS Genet 2014; 10: e1004494) to facilitate discovery of an association between a variant and a phenotype. (Gudbjartsson D F, et al., Nat Genet 2015; 47: p. 435-44; Consortium U K, et al., Nature 2015; 526: 82).

Such efforts have facilitated the discovery of a few therapeutic targets. For example, loss of function (LoF) mutations have been identified in the PCSK9 gene (Kathiresan, S. and C. Myocard Infarction, N Engl J Med 2008; 358: 2299) and in the APOC3 gene (Pollin T I, et al., Science 2008; 322: 1702) that are associated with favorable lipid profiles and reduced risk for coronary heart disease, and those discoveries have facilitated the development of therapeutics that target the products of those genes.

However, further elucidation of genetic factors that affect health and disease and the development of targeted therapeutics based on this information are needed to drive the implementation of precision medicine, and to identify more biological targets for pharmacological intervention. One approach for identifying putative biological targets is to statistically associate a variant of interest with a phenotype (or vice versa) in a large population of subjects for whom genetic variant and phenotype information is available (for example, Wellcome Trust Case Control Consortium, Nature 2007; 447: 661; Cohorts for Heart and Aging Research in Genomic Epidemiology Consortium, Circulation: Cardiovascular Genetics 2009; 2: 73). Large-scale sequencing of individuals with such phenotype-rich electronic health records provides an unprecedented opportunity to understand genetic variants and their effect on phenotypes. Conventional approaches, such as Genome Wide Association Studies (GWAS) and Exome Wide Association Studies (ExWAS), identify statistically significant associations that link genetic variants to the phenotype under study. Such associations often inspire hypotheses and investigations that aim to explain the physiological role of the corresponding genes. In contrast to single-trait associations, a pattern of associations to many phenotypes from multiple independent variants within the same gene may shed additional light on its biological role. Agnostic evaluation of such association signatures can potentially connect lesser understood genes to well-studied ones and reveal novel functional relationships.

### BRIEF SUMMARY

Disclosed are methods comprising determining, for each of a plurality of phenotypes, an association score indicative of an association between at least one variant of each gene of a plurality of genes and a phenotype of the plurality of phenotypes, determining, for each gene of the plurality of genes, based on the association scores, a gene-level association score indicative of a representative association between each gene of the plurality of genes and each phenotype of the plurality of phenotypes, generating, based on the gene-level association scores, a gene-phenotype score matrix, wherein the gene-phenotype score matrix comprises, for each gene of the plurality of genes, the gene-level association score for each phenotype of the plurality of phenotypes, receiving a selection of a gene-of-interest, determining, based on the selection, in the gene-phenotype score matrix, gene-level association scores of the gene-of-interest, determining, in the gene-phenotype score matrix, one or more genes associated with gene-level association scores similar to the gene-level association scores of the gene-of-interest, and identifying a gene of the one or more genes as a gene associated with the gene-of-interest.

Disclosed are methods comprising determining, for each of a plurality of phenotypes, an association score indicative of an association between at least one variant of each gene of a plurality of genes and a phenotype of the plurality of phenotypes, determining, for each gene of the plurality of genes, based on the association scores, a gene-level association score indicative of a representative association between each gene of the plurality of genes and each phenotype of the plurality of phenotypes, and generating, based on the gene-level association scores, a gene-phenotype score matrix, wherein the gene-phenotype score matrix comprises, for each gene of the plurality of genes, the gene-level association score for each phenotype of the plurality of phenotypes.

Disclosed are methods comprising receiving a selection of a gene-of-interest, determining, based on the selection, in a gene-phenotype score matrix, gene-level association scores of the gene-of-interest, wherein the gene-phenotype score matrix comprises, for each gene of a plurality of genes, a gene-level association score for each phenotype of a plurality of phenotypes, determining, in the gene-phenotype score matrix, one or more genes associated with gene-level association scores similar to the gene-level association scores of the gene-of-interest, and identifying a gene of the one or more genes as a gene associated with the gene-of-interest.

Disclosed are methods comprising generating, for each of a plurality of phenotypes, a variant-phenotype association data structure, determining, for each gene in the genotype-phenotype association data structures, a gene-level association score, generating, based on the gene-level association scores, a gene-phenotype score matrix data structure, and determining, based on a target gene and the gene-phenotype score matrix data structure, one or more genes associated with the target gene.

Disclosed are methods comprising administering a therapeutic agent to a subject, wherein the subject has been determined to have a specific set of phenotypes associated with a target gene, wherein the therapeutic agent alters expression of one or more genes associated with the target gene, and wherein the altered expression of one or more genes associated with the target gene provides a therapeutic effect to the subject.

Disclosed are apparatuses configured to perform any of the disclosed methods.

Disclosed are systems configured to perform any of the disclosed methods.

Disclosed are computer readable media having processor-executable instructions embodiment thereon configured to cause an apparatus to perform any of the disclosed methods.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows an example method.

FIG. 2 shows an example variant-phenotype association data structure.

FIG. 3 shows an example gene-level association data structure.

FIG. 4 shows an example gene-phenotype score matrix.

FIG. 5 shows an example method.

FIG. 6 shows an example method.

FIG. 7 shows a selection of a gene-of-interest in a gene-phenotype score matrix data structure.

FIG. 8 shows an example method of applying Principal Component Analysis (PCA) to a gene-phenotype score matrix.

FIGS. 9A-D show average F1 scores associated with various methods for identifying relevant genes.

FIG. 10 shows an example operating environment.

FIG. 11 shows an example method.

FIG. 12 shows an example method.

FIG. 13 shows an example method.

FIG. 14 shows an example method.

### DETAILED DESCRIPTION

The disclosed method and compositions may be understood more readily by reference to the following detailed description of particular embodiments and the Example included therein and to the Figures and their previous and following description.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, reference to “a TCR” includes a plurality of such TCRs, reference to “the dextramer” is a reference to one or more dextramers and equivalents thereof known to those skilled in the art, and so forth.

The term “subject” or “donor” may refer to an animal, such as a mammalian species (preferably human) or avian (e.g., bird) species. More specifically, a subject or donor can be a vertebrate, e.g., a mammal such as a mouse, a primate, a simian or a human. Animals include farm animals, sport animals, and pets. A subject or donor can be a healthy individual, an individual that has symptoms or signs or is suspected of having a disease or a predisposition to the disease, or an individual that is in need of therapy or suspected of needing therapy. In some embodiments, the subject donor is human, such as a human who has, or is suspected of having, cancer.

The term “barcode,” as used herein, generally refers to a label that may be attached to a molecule (e.g., dextramer, cell) to convey information about the molecule. For example, a DNA barcode can be a polynucleotide sequence attached to each dextramer and a common sequencing barcode can be a polynucleotide sequence attached during sequencing. This barcode can then be sequenced. The presence of the same barcode on multiple sequences may provide information about the origin of the sequence. For example, a barcode may indicate that the sequence came from a particular dextramer. A barcode can also indicate that a sequence came from a particular cell/dextramer combination.

As used herein, the terms “sequencing” or “sequencer” refer to any of a number of technologies used to determine the sequence of a biomolecule, e.g., a nucleic acid such as DNA or RNA. Exemplary sequencing methods include, but are not limited to, targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performed by a gene analyzer such as, for example, gene analyzers commercially available from Illumina or Applied Biosystems.

A “polynucleotide”, “nucleic acid”, “nucleic acid molecule”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Oligonucleotides often range in size from a few monomeric units, e.g. 3-4, to hundreds of monomeric units. Whenever a polynucleotide is represented by a sequence of letters, such as “ATGCCTG,” it will be understood that the nucleotides are in 5′→3′ order from left to right and that “A” denotes adenosine, “C” denotes cytosine, “G” denotes guanosine, and “T” denotes thymidine, unless otherwise noted. The letters A, C, G, and T may be used to refer to the bases themselves, to nucleosides, or to nucleotides comprising the bases, as is standard in the art.

The term “DNA (deoxyribonucleic acid)” refers to a chain of nucleotides comprising deoxyribonucleosides that each comprise one of four nucleobases, namely, adenine (A), thymine (T), cytosine (C), and guanine (G). The term “RNA (ribonucleic acid)” refers to a chain of nucleotides comprising four types of ribonucleosides that each comprise one of four nucleobases, namely; A, uracil (U), G, and C. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “nucleotide sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

As used herein, the term “genetic variant” or “variant” refers to a nucleotide sequence in which the sequence differs from the sequence most prevalent in a population, for example by one nucleotide, in the case of the SNPs described herein. For example, some variations or substitutions in a nucleotide sequence alter a codon so that a different amino acid is encoded resulting in a genetic variant polypeptide. The term “genetic variant,” can also refer to a polypeptide in which the sequence differs from the sequence most prevalent in a population at a position that does not change the amino acid sequence of the encoded polypeptide (i.e., a conserved change). Genetic variant polypeptides can be encoded by a risk haplotype, encoded by a protective haplotype, or can be encoded by a neutral haplotype. Genetic variant polypeptides can be associated with risk, associated with protection, or can be neutral.

Non-limiting examples of genetic variants include frameshift, stop gained, start lost, splice acceptor, splice donor, stop lost, inframe indel, missense, splice region, synonymous and copy number variants. Non-limiting types of copy number variants include deletions and duplications.

“Optional” or “optionally” means that the subsequently described event, circumstance, or material may or may not occur or be present, and that the description includes instances where the event, circumstance, or material occurs or is present and instances where it does not occur or is not present.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

“Exemplary” means “an example of” and is not intended to convey an indication of a preferred or ideal configuration. “Such as” is not used in a restrictive sense, but for explanatory purposes.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

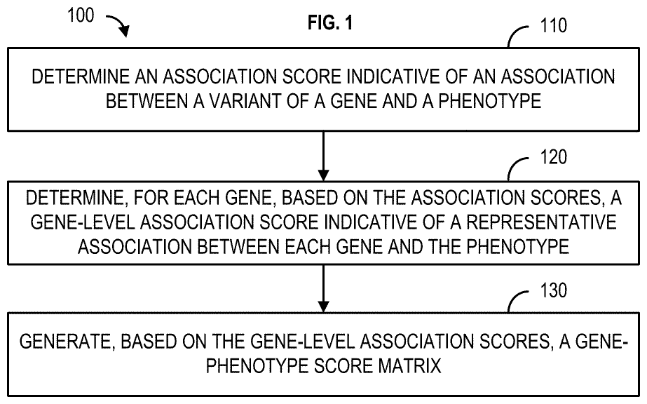

As shown in FIG. 1, a method 100 is disclosed for analyzing results from genome-wide association study (GWAS) and/or an exome-wide association study (ExWAS). The method 100 may comprise, at step 110, determining an association score indicative of an association between a variant of a gene and a phenotype. The method 100 may comprise, at step 120, determining, for each gene, based on the association scores, a gene-level association score indicative of a representative association between each gene and the phenotype. The method 100 may comprise, at step 130, generating, based on the gene-level association scores, a gene-phenotype score matrix.

At step 110, determining an association score indicative of an association between a variant of a gene and a phenotype may comprise conducting a statistical association analysis associated with a GWAS and/or an ExWAS. In an aspect, the statistical association analysis that is performed is a GWAS statistical analysis (van der Sluis S, et al., PLOS Genetics 2013; 9: e1003235; Visscher P M, et al., Am J Hum Genet 2012; 90: 7). In a GWAS analysis, one determines what genes or genetic variants are associated with a phenotype of interest. In one aspect, the genetic variant data are obtained from genomic sequencing of the subjects for whom genetic variant and phenotype data are contained in the system. In another aspect, the genetic variant data are obtained from exome (for example, whole exome) sequencing of the subjects for whom genetic variant and phenotype data are contained in the system.

In another aspect, the statistical association analysis that is performed is an ExWAS statistical analysis (Majewski, J., et al. (2011). What can exome sequencing do for you? J. Med. Genet. 48, 580-589). ExWAS naturally expand on findings from genome-wide association studies through their exploration of the functional region of the genome. ExWAS have been extensively used to dissect the genetic architecture of complex diseases and quantitative traits (Lee, S., et al. (2014). Rare-variant association analysis: study designs and statistical tests. Am. J. Hum. Genet. 95, 5-23). Exonic variants, particularly loss-of-function variants, tend to show the most dramatic effect sizes, yielding the greatest power for detection. Recent evidence on lipid traits provides support that rare variants can be ancestry-specific (Lu, X., et al. (2017). Exome chip meta-analysis identifies novel loci and East Asian-specific coding variants that contribute to lipid levels and coronary artery disease. Nat. Genet. 49, 1722-1730.). Therefore, examining exonic variants across diverse ancestry groups augments the identification of novel loci.

In an aspect, a result of a GWAS and/or ExWAS, statistical analysis may comprise one or more summary statistics. In an embodiment, the one or more summary statistics may be derived from results of a regression analysis. The regression analysis may include, for example, linear regression, mixed linear regression, multiple linear regression, logistic regression, multiple logistic regression, combinations thereof, and the like. The one or more summary statistics may be referred to as association scores. The association scores indicate a level of association between a variant and a phenotype and/or between a gene and a phenotype. The association scores may include, for example, a Z-score, a statistic based on Fisher's method, a rank sum statistic, a p-value, combinations thereof, and the like. In an aspect, GWAS and ExWAS results may be determined through performance of a GWAS or ExWAS study and performance of the statistical association analysis or may be obtained from publically accessible websites, published supplementary material, or through collaborations with investigators.

In an embodiment, data derived from a phenome-wide association study (PheWAS) statistical analysis (Denny J C, et al., Nature Biotechnol 2013; 31: 1102) may be subjected to one or more statistical techniques to derive data that may be used with the disclosed methods and systems. In a PheWAS study, one determines phenotypes that are associated with one or more genes or genetic variants of interest. In PheWAS, associations between one or more specific genetic variants and one or more physiological and/or clinical outcomes and phenotypes can be identified and analyzed. In an aspect, algorithms can be utilized to analyze electronic medical record (EMR) and electronic health record (EHR) data. In another aspect, data collected in observational cohort studies can be analyzed. Data derived from a PheWAS does not generally include an association score indicating an association of a phenotype to a variant, rather than a variant to a phenotype. In an embodiment, one or more statistical techniques may be applied to PheWAS data to derive an association score indicative of a level of association between a variant and a phenotype and/or between a gene and a phenotype. The association scores so derived from PheWAS data may be used with the methods and systems described herein.

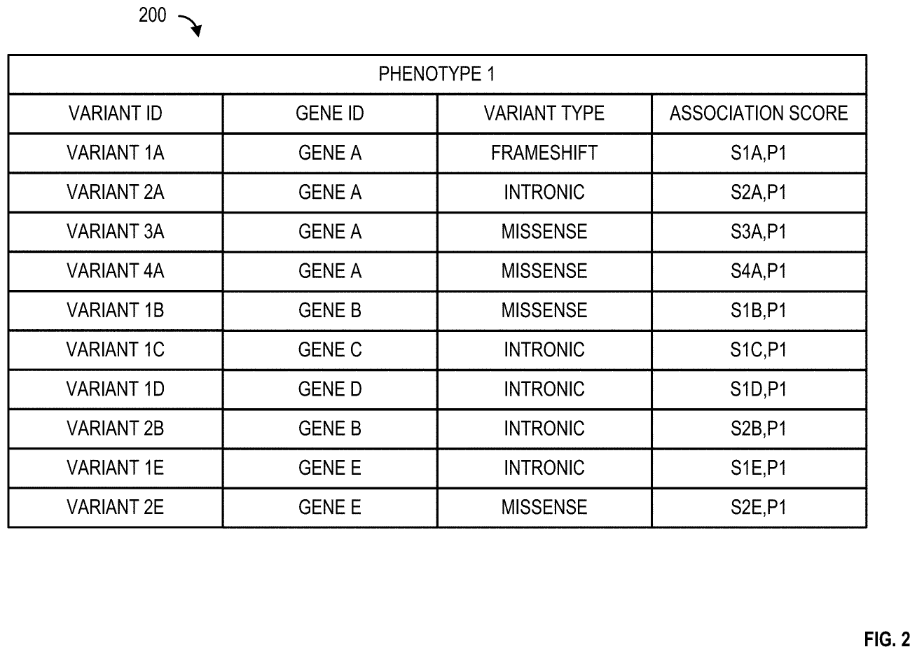

The association scores, whether determined or otherwise acquired, may be stored in a variant-phenotype association data structure 200 as shown in FIG. 2. Any suitable data structure may be used. A variant-phenotype association data structure 200 may be generated for each phenotype that was part of the GWAS and/or ExWAS. The variant-phenotype association data structure 200 may be stored and/or manipulated within a memory of a computing device (e.g., the memory system 1010). The variant-phenotype association data structure 200 may comprise one or more columns and one or more rows, resulting in one or more cells at an intersection of a row and a column. In an embodiment, the variant-phenotype association data structure 200 may comprise a logical table. The logical table may be generated such that the logical table comprises a plurality of logical rows, each said logical row including a variant identifier to identify each said logical row, each said logical row corresponding to a record of information. The logical table may be generated such that the logical table comprises a plurality of logical columns intersecting said plurality of logical rows to define a plurality of logical cells, each said logical column including a column identifier to identify each said logical column. Each of the plurality of logical cells may comprise data associated with the variant identifier and corresponding to the column identifier. The column identifiers may comprise one or more of, “VARIANT ID,” “GENE ID,” “VARIANT TYPE,” and/or “ASSOCIATION SCORE.” In an aspect, additional column identifiers are contemplated. For example, additional association score column identifiers may be used to support a plurality of association scores. The variant-phenotype association data structure 200 may comprise one or more rows for each gene, as each gene may have one or more variants. The ASSOCIATION SCORE column of the variant-phenotype association data structure 200 indicates a score indicated a measure of association of a variant to the phenotype. For example, in the variant-phenotype association data structure 200, Variant 1A of Gene A has an association score with Phenotype 1 (P1) represented by way of example as S1A,P1. In an embodiment, S1A,P1 may be a score, such as a Z-score, a statistic based on Fisher's method, a rank sum statistic, a p-value, combinations thereof, and the like. In an embodiment, the score may be derived from results of a regression analysis. The regression analysis may include, for example, linear regression, mixed linear regression, multiple linear regression, logistic regression, multiple logistic regression, combinations thereof, and the like. In an embodiment, a plurality of variant-phenotype association data structure 200 may be generated, with one phenotype per variant-phenotype association data structure.

Returning to FIG. 1, the method 100 may comprise, at step 120, determining, for each gene, based on the association scores, a gene-level association score indicative of a representative association between each gene and the phenotype. The determination of the gene-level association score may comprise determining the highest value (e.g., maximum), or the lowest value (e.g., minimum), association score for a given gene. The variant-phenotype association data structure 200 may be used to determine which association score for a given gene is the highest, or the lowest, depending on what the association score represents. In an embodiment, the variant-phenotype association data structure 200 comprises more than one ASSOCIATION SCORE column (e.g., z-score and p-value). In such embodiments, a determination may be made regarding which association score to use to determine the gene-level association score.

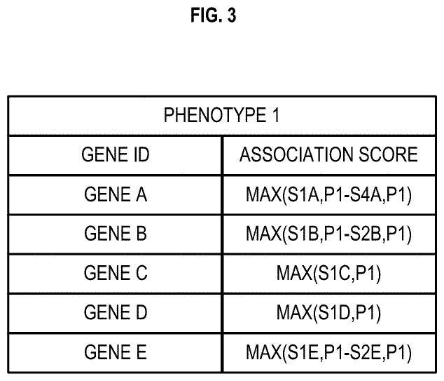

The gene-level association scores may be stored in a gene-level association data structure 300 as shown in FIG. 3. Any suitable data structure may be used. The gene-level association data structure 300 may be stored and/or manipulated within a memory of a computing device (e.g., the memory system 1010). The gene-level association data structure 300 may comprise one or more columns and one or more rows, resulting in one or more cells at an intersection of a row and a column. In an embodiment, the gene-level association data structure 300 may comprise a logical table. The logical table may be generated such that the logical table comprises a plurality of logical rows, each said logical row including a gene identifier to identify each said logical row, each said logical row corresponding to a record of information. The logical table may be generated such that the logical table comprises a one or more logical columns intersecting said plurality of logical rows to define a plurality of logical cells, each said logical column including a column identifier to identify each said logical column. Each of the plurality of logical cells may comprise data associated with the gene identifier and corresponding to the column identifier. The column identifier may comprise one or more of, “GENE ID,” and “ASSOCIATION SCORE.” In an aspect, additional column identifiers are contemplated. In contrast to the variant-phenotype association data structure 200, the gene-level association data structure 300 may comprise only one row for each gene, as the gene-level association score is a representative score associated with one variant of the gene. The ASSOCIATION SCORE column of the gene-level association data structure 300 indicates a maximum z-value for each gene in the variant-phenotype association data structure 200. A gene-level association data structure 300 may be generated for each variant-phenotype association data structure.

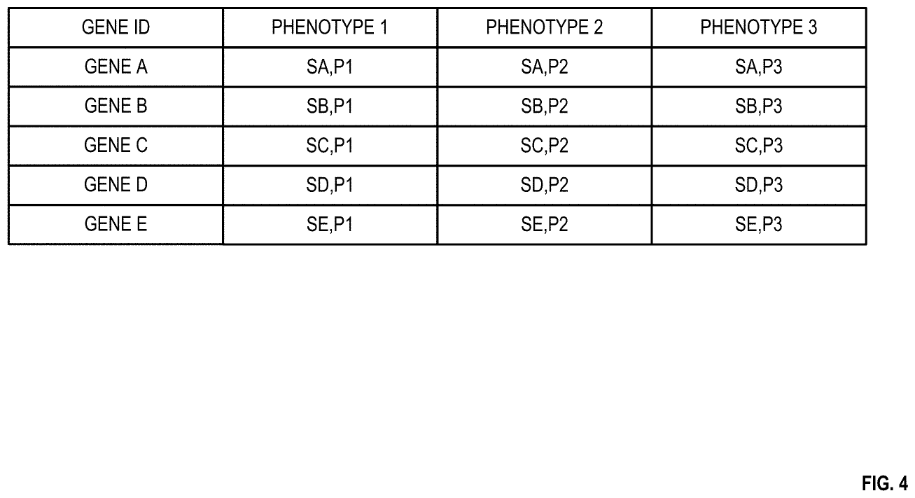

Returning to FIG. 1, the method 100 may comprise, at step 130, generating, based on the gene-level association scores, a gene-phenotype score matrix. Generating the gene-phenotype score matrix may comprise accessing a plurality of gene-level association data structures and assembling the plurality of gene-level association data structures into the gene-phenotype score matrix. The gene-level association data structures, may be stored in a gene-phenotype score matrix data structure 400 as shown in FIG. 4. Any suitable data structure may be used. The gene-phenotype score matrix data structure 400 may be stored and/or manipulated within a memory of a computing device (e.g., the memory system 1010). The gene-phenotype score matrix data structure 400 may be configured to represent the gene-level association scores, for each gene and each phenotype that was part of the GWAS and/or ExWAS.

The gene-phenotype score matrix data structure 400 indicates the association scores between genes and phenotypes and can be used to make recommendations. For example, each gene may have a corresponding row and each phenotype may have a corresponding column in the gene-phenotype score matrix data structure 400, and the association score between any given gene and phenotype may be indicated by the value in the gene-phenotype score matrix data structure 400 corresponding to the intersection of the given gene row and the given phenotype column. The gene-phenotype score matrix data structure 400 includes numerous genes and phenotypes and thus can be very large. For example, if 10,000 genes and 10,000 phenotypes are in the gene-phenotype score matrix data structure 400, the gene-phenotype score matrix data structure 400 may have dimensions of 10,000 by 10,000, far exceeding the capacity for human mental processing. Processing may be performed more quickly and with fewer resources if the gene-phenotype score matrix data structure 400 is reduced in size, as described herein.

The gene-phenotype score matrix data structure 400 may comprise one or more columns and one or more rows, resulting in one or more cells at an intersection of a row and a column. In an embodiment, the gene-phenotype score matrix data structure 400 may comprise a logical table. The logical table may be generated such that the logical table comprises a plurality of logical rows, each said logical row including a gene identifier to identify each said logical row, each said logical row corresponding to a record of information. The logical table may be generated such that the logical table comprises a plurality of logical columns intersecting said plurality of logical rows to define a plurality of logical cells, each said logical column including a column identifier to identify each said logical column. Each of the plurality of logical cells may comprise data associated with the gene identifier and corresponding to the phenotype identifier. The column identifiers may comprise one or more of, “GENE ID,” “PHENOTYPE 1,” “PHENOTYPE 2,” and/or “PHENOTYPE 3.” In an aspect, additional column identifiers are contemplated, specifically, one column identifier for each phenotype. The gene-phenotype score matrix data structure 400 may comprise one row for each gene. The PHENOTYPE N column of the gene-phenotype score matrix data structure 400 indicates the gene-level score for the gene in the row and the phenotype in the column, indicating a measure of association of the gene (by way of a variant) to the phenotype. For example, in the gene-phenotype score matrix data structure 400, Gene A has an association score with Phenotype 1 (P1) represented by way of example as SA,P1. In an embodiment, a single gene-phenotype score matrix data structure 400 may be generated to represents the results of the GWAS and/or ExWAS.

In an embodiment, the gene-phenotype score matrix may be filtered using one or more filters to remove pairs of variant-phenotype associations. The one or more filters may comprise a gene mapping filter, an association quality filters, linkage disequilibrium (LD) clumping, combinations thereof, and the like. The gene mapping filter may filter out variants that were not mapped to a protein coding gene or mapped to the intergenic regions were excluded. The association quality filter may filter out pairs of variant-phenotype associations with having a cell count less than a minimum threshold. The minimum threshold may be, for example, from, and/or including, about 10 to about 20 (e.g., a cell count<10). Linkage disequilibrium (LD) clumping may be applied at a threshold (e.g., r²=0.5) to remove variants that are in high LD with index variants for each phenotype under consideration. The threshold may be, for example, from, and/or including, from about 0 to about 1. In an embodiment, a higher threshold may lead to removal of variants that are in high LD. For a given phenotype, the index variants are variants with the most significant statistical associations (e.g., the smallest P-value) within a LD clump.

In an embodiment, one or more gene-phenotype score matrices (GPSM) may be generated.

A “best |Z| GPSM (X_{z})” defines a gene(i)-phenotype(j) score based on the maximum absolute value of Z-scores of associations between all variants annotated to gene(i) and phenotype(j).

A “normalized best |Z| GPSM (X_{z,N})” reassigns the value for each element in X_{z} by averaging the normalized values of the same element after applying quantile normalization to X_{z} along the row and column axes respectively.

A “best −log 10(Pval) GPSM (Xₚ)” defines a gene(i)-phenotype(j) score based on the maximum value of −log 10(Pval) from associations between all variants annotated to gene(i) and phenotype(j).

A “normalized best −log 10(Pval) (X_{p,N})” reassigns the value for each element in Xₚ by averaging the normalized values of the same element after applying quantile normalization to Xₚ along the row and column axes respectively.

The one or more gene-phenotype score matrices may be stored as one or more gene-phenotype score matrix data structures.

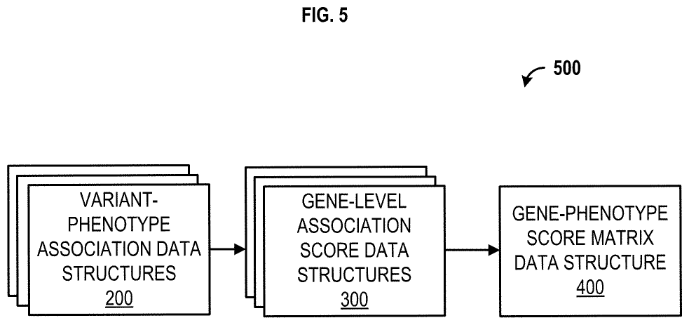

FIG. 5 shows a data flow for generating a gene-phenotype score matrix. A plurality of variant-phenotype association data structures 200 are generated, one for each phenotype. The variant-phenotype association data structures 200 are analyzed to determine a gene-level association score for each gene in each variant-phenotype association data structure 200 and are used to generate a plurality of gene-level association score data structures 300. Finally, the plurality of gene-level association score data structures 300 are used to generate the gene-phenotype score matrix data structure 400 which represents the gene-level association scores for each gene and each phenotype.

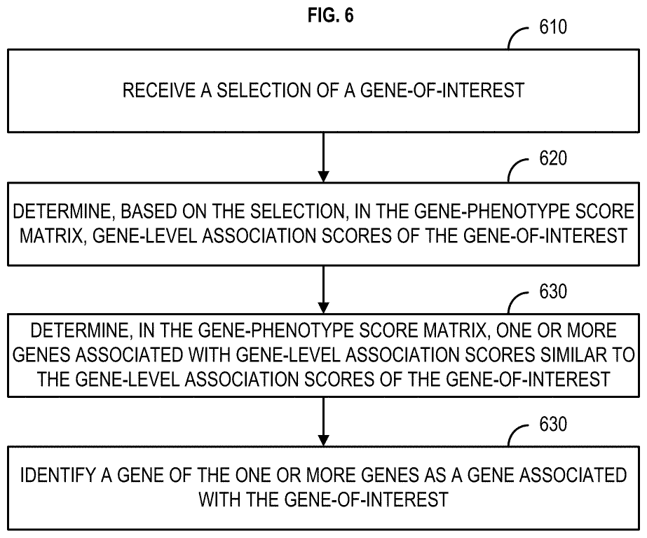

Once generated, the gene-phenotype score matrix data structure 400 may be used to determine unique associations amongst one or more genes. As shown in FIG. 6, a method 600 is disclosed for analyzing the gene-phenotype score matrix data structure. The method 600 may comprise, at step 610, receiving a selection of a gene-of-interest. The method 600 may comprise, at step 620, determining, based on the selection, in the gene-phenotype score matrix, gene-level association scores of the gene-of-interest. The method 600 may comprise, at step 630, determining, in the gene-phenotype score matrix, one or more genes associated with gene-level association scores similar to the gene-level association scores of the gene-of-interest. The method 600 may comprise, at step 640, identifying a gene of the one or more genes as a gene associated with the gene-of-interest.

At step 610, receiving a selection of a gene-of-interest may comprise receiving a gene identifier as an input, for example, from a user. A user may be presented with a list of genes present in the gene-phenotype score matrix as options for selection. In an aspect, a selection of a plurality of genes-of-interest may be received. For example, a user may select or otherwise input a gene identifier of “GENE B.”

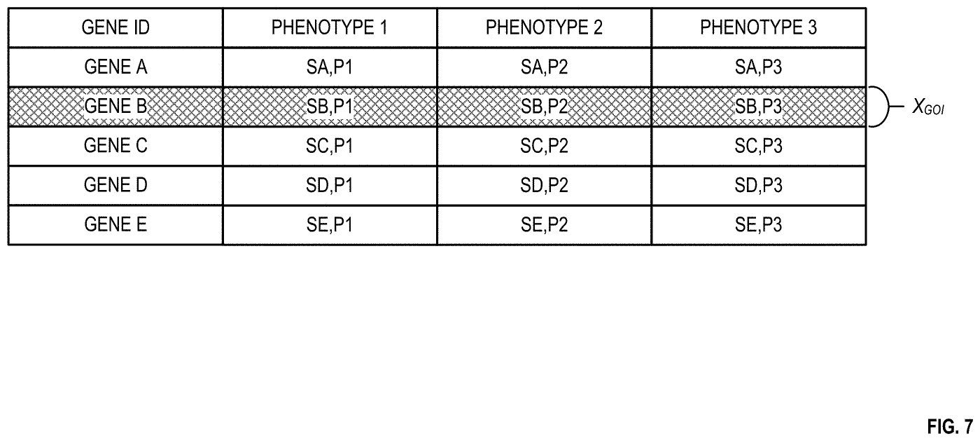

At 620, determining, based on the selection, in the gene-phenotype score matrix, gene-level association scores of the gene-of-interest may comprise determining, in the gene-phenotype score matrix, a gene-of-interest row containing the gene-level association scores of the gene-of-interest. For example, the gene-of-interest row may be determined by searching the gene-phenotype score matrix for a gene identifier that matches the gene-of-interest selected at step 610. Any suitable technique for searching the gene-phenotype score matrix may be used. As shown in FIG. 7, the gene-phenotype score matrix data structure 400 may be searched for a gene identifier received at step 610. A gene-of-interest row associated with a selection of gene identifier “GENE B” is indicated as “x_{GOI}”. The row for the gene-of-interest may be used to determine gene-level association scores for Gene B and any phenotypes that were part of a GWAS and/or ExWAS.

Returning to FIG. 6, the method 600 may comprise, at step 630, determining, in the gene-phenotype score matrix, one or more genes associated with gene-level association scores similar to the gene-level association scores of the gene-of-interest. In an embodiment, determining, in the gene-phenotype score matrix, the one or more genes associated with gene-level association scores similar to the gene-level association scores of the gene-of-interest may comprise determining, in the gene-phenotype score matrix, one or more rows containing gene-level association scores similar to the gene-level association scores in the gene-of-interest row. In an embodiment, the values of the rows of the gene-phenotype score matrix may be vectorized and a difference between a vector of the gene-of-interest row and each vector of the other rows of the gene-phenotype score matrix may be determined. One or more techniques can be applied to determine similarity between rows, for example, one or more correlation techniques (e.g., Pearson r, Spearman, Kendall's), a running Fisher algorithm, one or more clustering or neighbor graph techniques (e.g., PCA+clustering, t-SNE, UMAP), combinations thereof, and the like.

A generalized framework for determination of similarity between X_{GOI} and each of one or more other rows (xᵢ) as:

$$[ \begin{matrix}{x}_{GOI} \\ {x}_{i}\end{matrix} \} f \to {d}_{i} ] x ( n - 1 ) \to R$$

where dᵢ is as statistic that indicates similarity between gene i and the gene-of-interest and R is a ranking of n−1 genes based on similarity to the gene-of-interest.

In an embodiment, a principal component analysis (PCA) method may be used to determine one or more rows similar to the gene-of-interest row. A weighted PCA may be applied to the gene-phenotype score matrix. Each gene may be projected onto the top/first principal component (PC1). Candidate genes may be ranked based on their PC1 difference to the gene-of-interest (e.g., the smaller the PC1 difference, the more similar to the gene-of-interest).

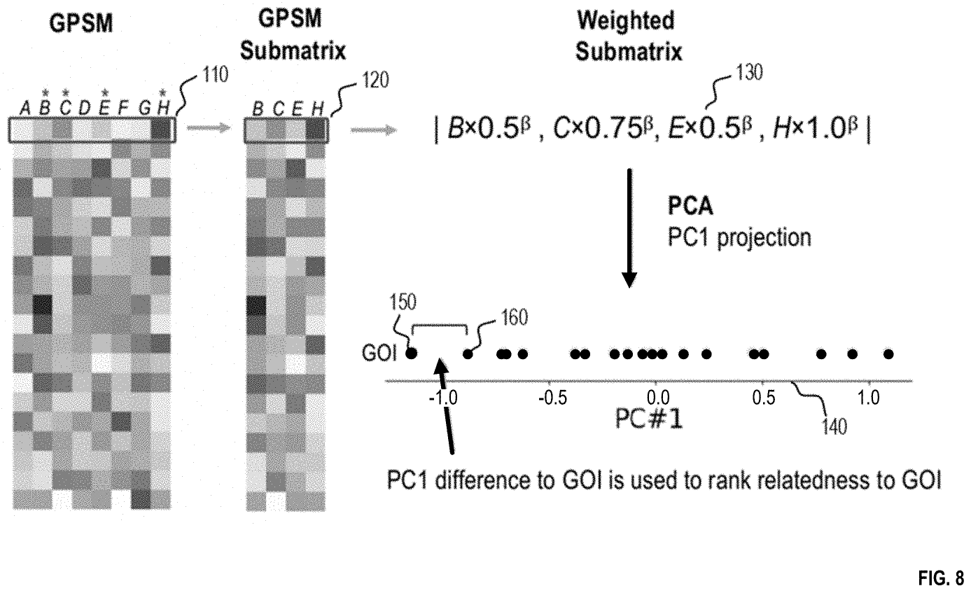

As shown in FIG. 8, in an embodiment, a gene-phenotype score matrix 810 may be reduced prior to application of PCA. A large gene-phenotype score matrix 810 may present several technical problems. The gene-phenotype score matrix 810 may require a significant amount of memory for storage and processing. It may also take a long time to load the gene-phenotype score matrix 810 into memory, such as when the gene-phenotype score matrix 810 is used in a distributed environment (e.g., the Internet). Matrix-reduction algorithms may be used to reduce the size of a large gene-phenotype score matrix 810. The reduced gene-phenotype score matrix (also referred to as a gene-phenotype score submatrix 820) may be generated according to a variety of techniques. In an embodiment, the gene-phenotype score matrix 810 may be reduced in size using, for example, a matrix decomposition algorithm, such as singular value decomposition (SVD).

In an embodiment, the gene-phenotype score submatrix 820 may be generated by first applying a threshold to the gene-level association scores in the gene-phenotype score matrix 810. Any column that contains gene-level association scores that do not satisfy the threshold may be removed from the gene-phenotype score matrix 810 to generate the gene-phenotype score submatrix 820.

As described above, each row of the gene-phenotype score matrix 810 (or submatrix 820) may be considered a vector. Principal component analysis (PCA) may be used to determine similarity between vectors. PCA involves a mathematical procedure that transforms a number of (possibly) correlated variables into a (smaller) number of uncorrelated variables called principal components. The first principal component accounts for as much of the variability in the data as possible, and each successive component accounts for as much of the remaining variability as possible.

A weighted submatrix 830 may be determined and PCA applied to the weighted submatrix 830. The result is a projection (PC1) 840. The projection 840 may be used to determine similarity between any gene vector (row) to the gene-of-interest vector (gene-of-interest row). The difference between any given vector in the projection 840 and the vector of the gene-of-interest 850 may be used to rank the relatedness of any given gene to the gene-of-interest. For example, a gene vector 860 has the least difference between the vector of the gene-of-interest 850. Accordingly, the gene associated with the gene vector 860 may be ranked as the gene most similar to the gene-of-interest.

In an embodiment, the present methods can rank gene-gene similarity using a weighted PCA method. Disclosed is a function ƒ(X, g, α, β) that inputs four variables to compute pairwise similarity between the gene of interest (g) and other n−1 candidate genes that are represented in the gene-phenotype score matrix (X). Here α and β are hyperparameters that determine the calculation outcome and can be optimized based on reference datasets as described herein.

Given X which has n rows each representing one gene and p columns each representing a phenotype, x_{i,j} denotes the score of i^{th} gene for j^{th} phenotype. xᵢ is a p-vector containing p scores of gene i for each phenotype and x_{g} represents the gene of interest (g). Similarity between xᵢ and x_{g} is computed based on the steps described below.

First, submatrix M may be extracted based on g and a. For a given X, there are n×p gene-phenotype scores and a is a percentile value that sets a predetermined threshold. Such a threshold is then used to select high-scoring phenotypes of g. For example, if x₇₅_{^{th}} is the 75^{th} percentile of all values in X, L is a list of k indices corresponding to phenotypes (columns) where g scores higher than x₇₅_{^{th}} (x_{g,j}>x₇₅_{^{th}}). A submatrix M={Xⱼ}_{j∈L} is extracted from X for downstream calculation. M has n rows and k columns.

Then, a vector of weight coefficients w and weighted submatrix N may be determined based on β. With the extracted submatrix M={Xⱼ}_{j∈L}, represents the j^{th} column containing n scores to phenotype j from each gene including g. The k-vector m_{g} represents the scores of g for the chosen k high-scoring phenotypes. To enable adjustable weighting of scores for the different phenotypes, a weight coefficient wⱼ∈[0,1] is calculated for each phenotype j by

$${w}_{j} = {( \frac{{m}_{g , j}}{max ( {m}_{g} )} )}^{β}$$

where predetermined β≥0. Consequently, the weighted submatrix N={wⱼ·Xⱼ}_{j∈L}.

A numerical difference on first principal component (PC1) between g and candidate genes may be determined. After obtaining the weighted submatrix N(n×p), N may be centered based on the mean of each column, computed the covariance matrix C, and obtained eigenmatrix V(p×p) by diagonalization. The numerical projection of the all n genes on the top/first principal component is calculated by

Y_{PC1}=NV₁

where V₁(p×1) is the first column of eigenmatrix V, Y_{PC1}(n×1) is a row in which y_{i,PC1} is the PC1 score of the i^{th} gene. The difference of PC1 scores between g and the remaining n−1 genes may be determined by d_{i,PC1}=y_{i,PC1}−y_{g,PC1}.

Gene-specific “bias” from empirical null simulation may be corrected. Various factors, such as gene size and tolerance to mutations, can bias PC1 scores of candidate genes and subsequent d_{i,PC1} regardless of the chosen gene of interest (g). To compensate for such biases, a correction factor bᵢ may be determined for each gene i based on input X, α, and β. Specifically, a random gene gₛ is first simulated by phenotype permutation from X, represented as a row vector x_{g}_{ₛ}(1×p). Using gₛ as the gene of interest, d_{i,PC1} is calculated for all n genes described in step 1-3. The calculation may be repeated for another 999 randomly simulated gₛ and the mean d̅_{i,PC1}=Σ₁¹⁰⁰⁰ d_{i,PC1}/1000 obtained. Subsequently, correction factor bᵢ of gene i can be computed as

$${b}_{i} = \frac{median {( {\overline{d}}_{i , P C 1} )}_{i ∈ n}}{{\overline{d}}_{i , P C 1}} .$$

Candidate genes may then be ranked based on their similarity to g. With a given set of X, g, α, β, the n−1 genes may be ranked based on their corrected PC1 differences to g in an ascending order where d_{i,PC1}^{c}=d_{i,PC1}·bᵢ for gene i. The significance of each d_{i,PC1}^{c} may be further estimated by computing a Z-score against a null distribution of 10,000 simulated genes.

Returning to FIG. 6, the method 600 may comprise, at step 640, identifying a gene of the one or more genes as a gene associated with the gene-of-interest. In an embodiment, identifying a gene of the one or more genes as a gene associated with the gene-of-interest may comprise determining a gene identifier associated with one or more gene vectors ranked in terms of relatedness/similarity to the gene-of-interest vector. The resulting list of gene identifiers may be output to an output device, such as a display device.

In some aspects, the one or more genes identified as being associated with a gene of interest can be determined to be in the same biological pathway as the gene of interest. For example, the identified genes may play a role in the same metabolic pathway, signaling pathway, or genetic pathway. Once it is determined that one or more identified genes could be associated with a gene of interest, expression of the one or more identified genes can be altered to determine the effects the altered expression can have on the gene of interest. Alternatively, the expression of the gene of interest can be altered to determine the effects it can have on the one or more identified genes. Altering expression can include increasing expression or decreasing expression. In some aspects, decreasing expression can comprise completely eliminating all gene expression, such as knocking out the gene.

In some aspects, the one or more identified genes are determined to be in a particular biological pathway. For example, if the one or more identified genes are determined to be in a disease pathway, the one or more identified genes can be targeted to help treat the disease. In some aspects, increased expression of the one or more identified genes can have a positive effect on the pathway/disease it was determined to be a part of. Thus, a therapeutic agent that directly or indirectly results in increased expression of the one or more identified genes can be used to provide a therapeutic effect, including treating the disease. In some aspects, a therapeutic agent can be, but is not limited to, a chemical compound, a peptide, a protein, an antibody, or a nucleic acid.

In some aspects, the one or more identified genes can be associated with a gene of interest and a specific set of phenotypes. Thus, if a subject was determined to have a specific set of phenotypes associated with a particular disease or condition, the one or more identified genes can be targeted to help treat at least that specific set of phenotypes. In some aspects, these are known as phenotype-specific treatments. Disclosed are methods comprising administering a therapeutic agent to a subject, wherein the subject has been determined to have a specific set of phenotypes associated with a target gene, wherein the therapeutic agent alters expression of one or more genes associated with the target gene, and wherein the altered expression of one or more genes associated with the target gene provides a therapeutic effect to the subject. The one or more genes associated with the target gene can be determined using the methods disclosed herein. In some aspects, the altered expression is an increase in expression of one or more genes associated with the target gene, wherein an increase in expression provides a therapeutic effect. In some aspects, the altered expression is a decrease in expression of one or more genes associated with the target gene, wherein a decrease in expression provides a therapeutic effect. For example, in a subject with heart failure, a specific set of phenotypes can be, but are not limited to, lung congestion, obesity, muscle weakness, and hypertension. Thus, the disclosed methods can be used to identify one or more genes associated with a gene of interest known to be involved in these phenotypes of heart failure. In some aspects, the one or more identified genes can be used to treat or provide a therapeutic effect to the specific heart failure phenotypes. In some aspects, a subject with heart failure not showing those specific phenotypes would not be treated with a therapeutic agent that targets the one or more identified genes associated with the specific set of phenotypes.

In some aspects, the function of a gene of interest that is hitherto uncharacterized can be inferred by genes that are similar to it when such genes are determined/known to be involved in a well-known biological mechanism. Thus, established experimental assays can be used to test hypotheses regarding the function of the gene of interest. For example, if multiple genes that are known to regulate lipid transport are associated to the gene of interest, in vitro assays that measures lipid transport can be performed in cells where the expression of the gene of interest is altered.

In some aspects, the gene of interest is chosen due to specific therapeutic interest in a certain set of phenotypes/conditions. If one or more identified genes that are associated to the gene of interest are molecular targets of existing therapeutics, the established connection between these identified genes/existing therapeutic targets and the gene of interest can motivate the repurposing of existing drugs. Here, existing therapeutics can be an antibody, a small molecule compound, a mRNA molecule, or other biologics.

In some aspects, the gene of interest is intended as a knockout target in certain model organisms, for example Mus musculus and Danio rerio, but homologs of the gene of interest do not exist in the chosen organism. If homologs of the one or more identified genes that are associated to the gene of interest exist in the chosen organism, the connections highlighted by the disclosed methods can propose alternative modeling targets.

In some aspects, the gene of interest that is useful for therapeutic intervention may not be amenable for modulation due to various reasons. In such cases, similar related genes identified by the disclosed methods may be more attractive targets amenable for therapeutic manipulation.

In some aspects, a group of identified genes, together with the gene of interest, can be treated as a gene set. The resulting gene set, which is derived from genomic association studies, can be used as an input dataset for gene set enrichment analysis to analyze gene expression data.

In some aspects, the gene of interest may enable diagnosis of a certain phenotype/disease based on the knowledge of connected genes, determined by the disclosed methods, and thus, facilitate discovery of new genes for known conditions.

In some aspects, the genetic variants in a gene of interest and other related genes determined by the disclosed methods may collectively inform on efficacy of drugs (pharmacogenomics). Thus identifying related genes can help inform studies along various lines of investigation.

Using the methods disclosed herein, gene-phenotype score matrices X from summary statistics of exome-wide association analyses of 4,273 phenotypes were generated. Association analyses were performed using whole exome sequences of 150,000 individuals with European ancestry and their corresponding electronic health records from UK Biobank.

Using ACAN, PCSK9, and LRP5 as genes of interest (GOIs), the disclosed methods ranked 19,012 genes based on predicted similarity to GOIs. The top 20 ranking candidate genes for each GOI are listed in the table below.

| | | |
|---|---|---|
| PCSK9 | ACAN | LRP5 |

| | | | | | |
|---|---|---|---|---|---|
| 1 | APOB | 1 | HAPLN3 | 1 | AXIN1 |
| 2 | LDLR | 2 | ADAMTS10 | 2 | WNT16 |
| 3 | USP24 | 3 | UQCC1 | 3 | CEPD1 |
| 4 | CELSR2 | 4 | CD79B | 4 | TPCN2 |
| 5 | APOE | 5 | SMARCD2 | 5 | PPP6R3 |
| 6 | SLC22A1 | 6 | EIF6 | 6 | IDUA |
| 7 | TM6SF2 | 7 | SLF2 | 7 | PTCH1 |
| 8 | POC5 | 8 | INO80E | 8 | PRKAG1 |
| 9 | IGF2R | 9 | LCORL | 9 | BLK |
| 10 | SLC22A3 | 10 | PKD1 | 10 | RUNX2 |
| 11 | NECTIN2 | 11 | ADAMTS17 | 11 | CKB |
| 12 | LPA | 12 | L3MBTL3 | 12 | SPTBN1 |
| 13 | TOMM40 | 13 | UHRF1BP1 | 13 | CCDC170 |
| 14 | DNM2 | 14 | OTUD4 | 14 | MEOX2 |
| 15 | SMARCA4 | 15 | HIST1H2BE | 15 | MTOR |
| 16 | ZPR1 | 16 | SCMH1 | 16 | DDN |
| 17 | ABCG5 | 17 | DOC2A | 17 | KREMEN1 |
| 18 | APOA5 | 18 | GDF5 | 18 | JAG1 |
| 19 | ASGR1 | 19 | GDF5OS | 19 | PKDCC |
| 20 | CEACAM19 | 20 | NOS3 | 20 | INSC |

To create a reference dataset which contains a list of genes related to a chosen gene of interest (g), human pathway annotations were extracted from Pathway Commons (www.pathwaycommons.org) and primary data was compiled from seven databases—Reactome, NCI Pathway Interaction Database, PANTHER, INOH, NetPath, PathBank, and Virtual Metabolic Human. After normalizing the gene identity, 3,826 pathways were compiled, collectively covering 10,814 genes. For each gene of interest (g), the union of all pathways to which it belongs was used as the final list of relevant genes R_{g}.

To examine the impact of different values of α and β on the ability of the disclosed methods to identify highly relevant genes when given a gene of interest (g), the top 100 ranking candidates (T¹⁰⁰) for 10,814 genes were compared and mean F1 scores (F̅1̅_{X,α,β}) over a range of α and β were calculated. Specifically,

$${\overline{F 1}}_{X , α , β} = \frac{{\sum }_{g = 1}^{g = 10 , 814} F {1}_{X , α , β , g}}{10 \text{,} 814}$$

where for each gene of interest (g)

$$F {1}_{X , α , β , g} = 2 \times \frac{\frac{{R}_{g} ⋂ {T}_{g}^{100}}{{R}_{g}} \times \frac{{R}_{g} ⋂ {T}_{g}^{100}}{{T}_{g}^{100}}}{\frac{{R}_{g} ⋂ {T}_{g}^{100}}{{R}_{g}} + \frac{{R}_{g} ⋂ {T}_{g}^{100}}{{T}_{g}^{100}}}$$

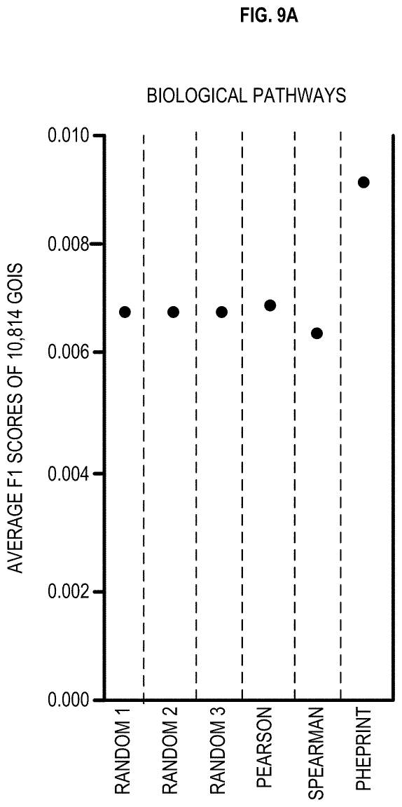

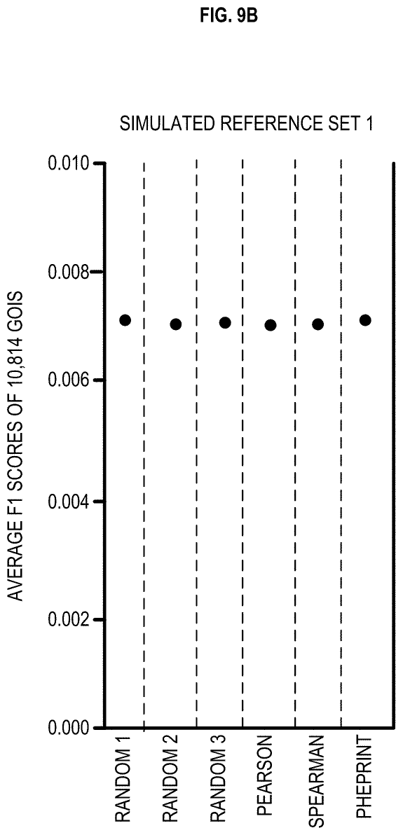

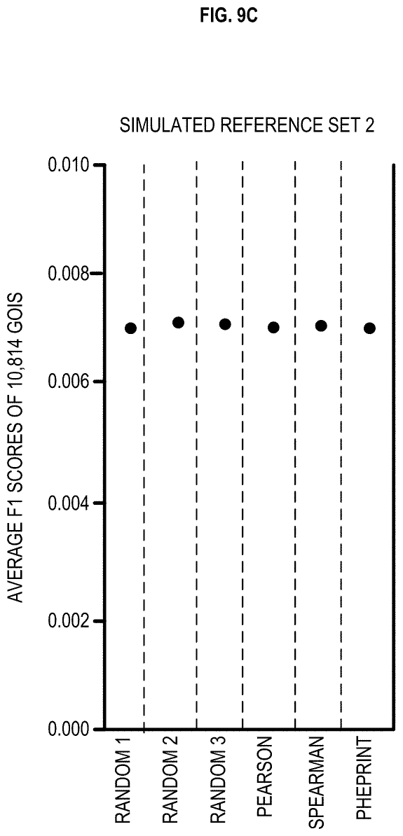

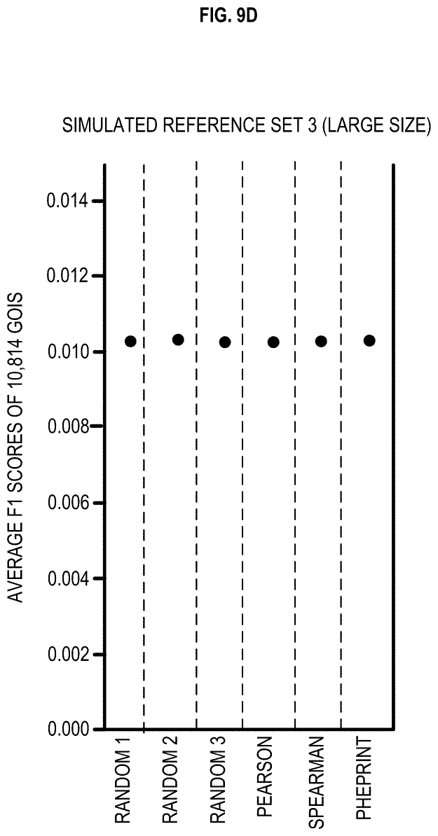

Additionally, the following methods were used to determine highly relevant genes: Pearson correlation, Spearman correlation, and the presently disclosed methods. Based on the top 100 ranking candidates from each method, an F1 score was calculated by comparing the top-100 ranking candidates to the corresponding reference set and the mean of F1 scores of 10,814 GOIs was subsequently computed. For each one of the ranking methods (random selection, Pearson correlation, Spearman Correlation, and the presently disclosed methods), average F1 scores were calculated against a reference set compiled from published biological pathways and three simulated reference sets whose members have no biological connections among each other. As shown in FIG. 9A-D, the average size of lists of relevant genes in simulated reference set 1 (FIG. 9B) and simulated reference set 2 (FIG. 9C) is 489, which is comparable to that of the biological reference set (FIG. 9A). As shown in FIG. 9D, average size of lists of relevant genes in simulated reference set 3 is 5,000. As shown in FIG. 9A-D, on average, the top 100 ranking candidates according to the present methods contained more pathway members for a given GOI than both correlation methods (as well as random selection) based on the current reference set. Similar trends are consistent for average F1 scores calculated from both top 20 and 50 candidates. These results demonstrate that biologically related genes, often called a pathway, can be mapped agnostically from human genetic association results with sufficient scale in both sample size and phenotype diversity. However, identification of these related genes from GWAS/ExWAS association results requires custom methods that are appropriate for these types of data, which can be noisy and incomplete. As shown in FIG. 9A, classical correlation methods such as Pearson or Spearman do not perform well in comparison to the presently disclosed methods. Based on the current reference set, identifying relevant genes to the gene of interest using Pearson or Spearman correlation is comparable to or worse than random selection, undermining the goal of the exercise. The methods disclosed thus represent a technological improvement over existing technology for identifying biologically significant similarity between genes. Such improvement directly impacts therapeutic treatments that may be administered to a subject on the basis of gene and/or biological pathway similarities.

To highlight that the present methods are particularly better at extracting meaningful biological relationships from association, average F1 score of 10,814 GOIs for each ranking method were also calculated against three simulated reference sets that are randomly synthesized without any biological foundation. As shown in FIG. 9B-D, the present methods perform equivalent to other correlation methods as well as random selection against the three simulated reference sets that are randomly synthesized without any biological foundation.

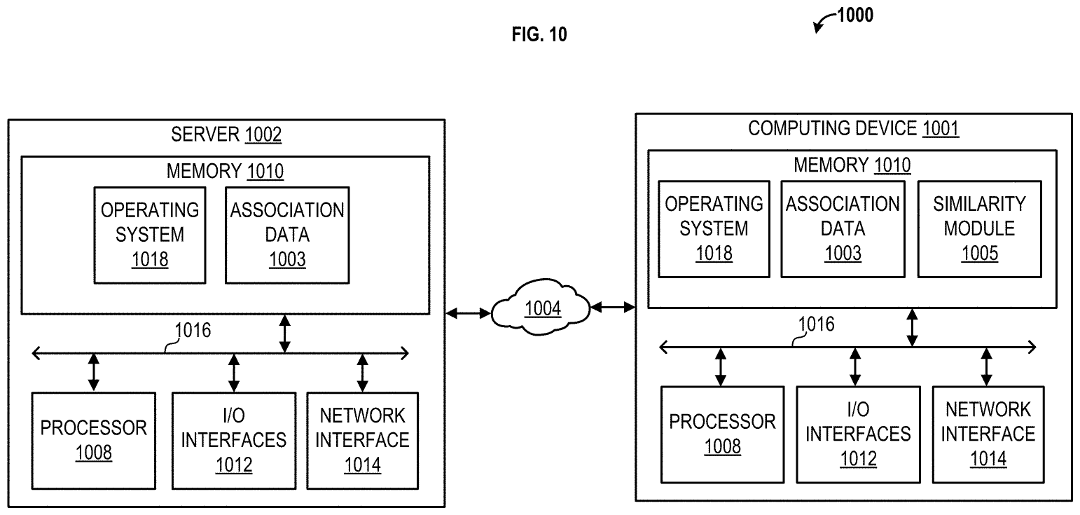

FIG. 10 is a block diagram depicting an environment 1000 comprising non-limiting examples of a computing device 1001 and a server 1002 connected through a network 1004. In an aspect, some or all steps of any described method may be performed on a computing device as described herein. The computing device 1001 can comprise one or multiple computers configured to store one or more of association data 1003 (e.g., GWAS and/or ExWAS association results, variant-phenotype association data structures, gene-level association score data structures, gene-phenotype score matrix data structure, and the like), a similarity module 1005 (e.g., software configured for performing any of the disclosed methods), and the like. The server 1402 can comprise one or multiple computers configured to store additional association data 1003. Multiple servers 1002 can communicate with the computing device 1001 via the through the network 1004. In an embodiment, the server 1002 may comprise a repository for data generated by a GWAS and/or an ExWAS.

The computing device 1001 and the server 1002 can be a digital computer that, in terms of hardware architecture, generally includes a processor 1008, memory system 1010, input/output (I/O) interfaces 1012, and network interfaces 1014. These components (1008, 1010, 1012, and 1014) are communicatively coupled via a local interface 1016. The local interface 1016 can be, for example, but not limited to, one or more buses or other wired or wireless connections, as is known in the art. The local interface 1016 can have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 1008 can be a hardware device for executing software, particularly that stored in memory system 1010. The processor 1008 can be any custom made or commercially available processor, a central processing unit (CPU), an auxiliary processor among several processors associated with the computing device 1001 and the server 1002, a semiconductor-based microprocessor (in the form of a microchip or chip set), or generally any device for executing software instructions. When the computing device 1001 and/or the server 1002 is in operation, the processor 1008 can be configured to execute software stored within the memory system 1010, to communicate data to and from the memory system 1010, and to generally control operations of the computing device 1001 and the server 1002 pursuant to the software.

The I/O interfaces 1012 can be used to receive user input from, and/or for providing system output to, one or more devices or components. User input can be provided via, for example, a keyboard and/or a mouse. System output can be provided via a display device and a printer (not shown). I/O interfaces 1012 can include, for example, a serial port, a parallel port, a Small Computer System Interface (SCSI), an infrared (IR) interface, a radio frequency (RF) interface, and/or a universal serial bus (USB) interface.

The network interface 1014 can be used to transmit and receive from the computing device 1001 and/or the server 1002 on the network 1004. The network interface 1014 may include, for example, a 10BaseT Ethernet Adaptor, a 100BaseT Ethernet Adaptor, a LAN PHY Ethernet Adaptor, a Token Ring Adaptor, a wireless network adapter (e.g., WiFi, cellular, satellite), or any other suitable network interface device. The network interface 1014 may include address, control, and/or data connections to enable appropriate communications on the network 1004.

The memory system 1010 can include any one or combination of volatile memory elements (e.g., random access memory (RAM, such as DRAM, SRAM, SDRAM, etc.)) and nonvolatile memory elements (e.g., ROM, hard drive, tape, CDROM, DVDROM, etc.). Moreover, the memory system 1010 may incorporate electronic, magnetic, optical, and/or other types of storage media. Note that the memory system 1010 can have a distributed architecture, where various components are situated remote from one another, but can be accessed by the processor 1008.

The software in memory system 1010 may include one or more software programs, each of which comprises an ordered listing of executable instructions for implementing logical functions. In the example of FIG. 10, the software in the memory system 1010 of the computing device 1001 can comprise the association data 1003, the similarity module 1005, and a suitable operating system (O/S) 1018. In the example of FIG. 10, the software in the memory system 1010 of the server 1002 can comprise, the association data 1003, and a suitable operating system (O/S) 1018. The operating system 1018 essentially controls the execution of other computer programs and provides scheduling, input-output control, file and data management, memory management, and communication control and related services.

The association data 1003 (e.g., the gene-phenotype score matrix data structure 400) may be represented as a multi-dimensional array (e.g., an array of one-dimensional arrays. When a given matrix element (e.g., association score) is being processed (e.g., sorted), its value and associated information, or a pointer to its value and associated information, moves to and from various memory locations and array registers. An array register or simply register, as used herein, is a memory circuit capable of storing one or more bits or words of data. The matrix data (which include matrix elements of the matrix) are stored in the memory system 1010 in any one of a variety of matrix-storage formats; that is, formats for storing zero matrix elements and/or non-zero matrix elements of the matrix in the memory system 1010 and for locating such stored matrix elements. Examples of such matrix-storage formats include a compressed sparse row (CSR) format, a compressed sparse column (CSC) format, and a coordinate format. In the CSR format, the matrix element data and column index are stored as pairs in an array format. Another array stores a row start address for each column; these pointers can be used to look up the memory locations in which the rows are stored. In the CSC format, the matrix element data value and row index are stored as pairs in an array format. Another array stores a column start address for each row. The coordinate format stores data related to a matrix element together in array format, such related data including the matrix element data value, row index, and column index. Storing the association data (e.g., the gene-phenotype score matrix data structure 400) in such a fashion represents a departure from how traditional GWAS, ExWAS, and/or PheWAS association data is stored. A direct result of such storage is increased processing speed and efficiency, which represents an improvement over state of the art techniques for assessing gene similarity.

For purposes of illustration, application programs and other executable program components such as the operating system 1018 are illustrated herein as discrete blocks, although it is recognized that such programs and components can reside at various times in different storage components of the computing device 1001 and/or the server 1002. An implementation of the similarity module 1005 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

In an embodiment, the similarity module 1005 may be configured to perform some or all of the operations for gene similarity analysis operations and may store intermediate results to the memory system 1010 before performing post processing to generate an output vector (e.g., a gene associated with, related to, similar to, and the like, to a gene-of-interest). That is, the system 1000 receives, or otherwise determines, an initial input vector for a gene (or genes) of interest that is provided as input to the similarity module 1005. In addition, the system 1000 may generate, retrieve, or otherwise determine variant-phenotype association data structures, gene-level association score data structures, and/or a gene-phenotype score matrix data structure (the association data 1003) via the similarity module 1005. The similarity module 1005 comprises logic that operates on the input vector and the gene-phenotype score matrix data structure to perform gene similarity analysis operations involving iterations of matrix vector operations to identify genes in the gene-phenotype score matrix data structure that are related to the gene (or genes) specified in the input vector.

It should be appreciated that the input vector may comprise any number of genes and in general can range from 1 gene to hundreds, or thousands of genes. In some illustrative embodiments, the input vector may be one of a plurality of input vectors that together comprise an N*M input matrix. Each input vector of the N*M input matrix may be handled separately during gene similarity analysis operations as separate matrix vector operations, for example. The gene-phenotype score matrix data structure may represent an N*N square matrix which may comprise hundreds or thousands of genes and/or phenotypes and their scores.

The similarity module 1005 may require multiple iterations to perform a gene similarity analysis operation. For example, a concept analysis operation may utilize a plurality of iterations of the matrix vector operations to achieve a converged result, although more or less iterations may be used. With the gene-phenotype score matrix data structure representing up to hundreds or thousand of genes, phenotypes and scores, and the input vector(s) representing potentially hundreds or thousand of genes, the processing resources required to perform these multiple iterations is quite substantial.

The results generated by the similarity module 1005 comprise one or more output vectors specifying the genes in the gene-phenotype score matrix data structure that are related to the gene(s) in the input vector. Each non-zero value in the one or more output vectors indicates a related gene. The value itself is indicative of the strength of the relationship between the genes. The result may be stored in the memory system 1010 and can be very large due to potentially large scale input matrix and vector(s).

As part of a post processing, the similarity module 1005 retrieves the output vector results stored in the memory system 1010 and performs a ranking operation on the output vector results. The ranking operation essentially ranks the genes according to strength values in the output vector such that the highest ranked genes are ranked higher than the other genes. The similarity module 1005 then outputs a final N-element output vector representing a ranked listing of the genes related to the gene(s) of interest.

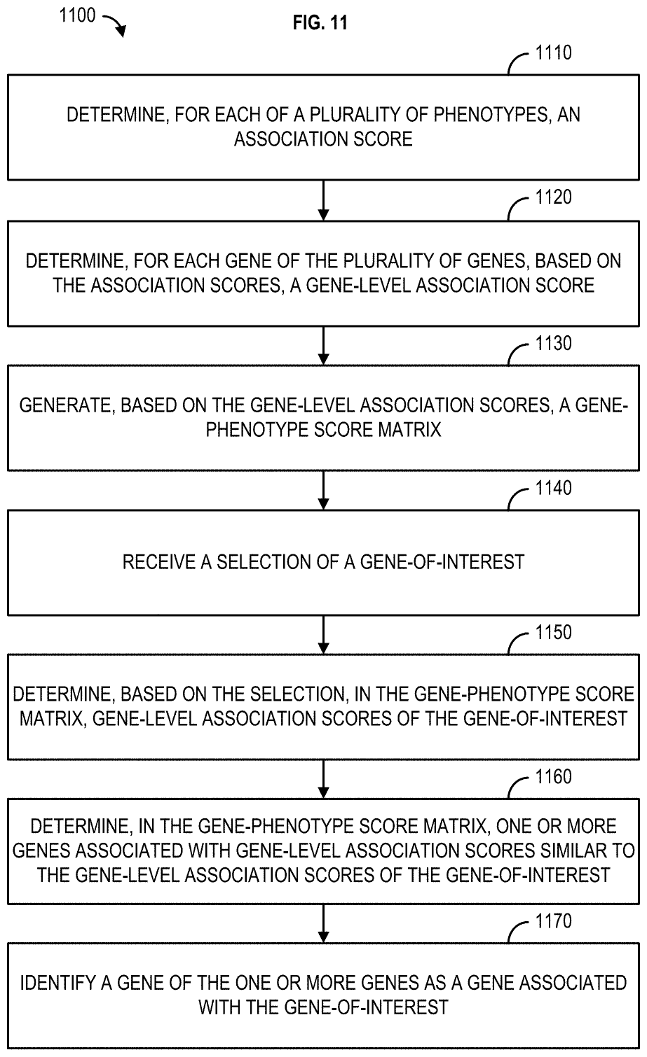

In an embodiment, the similarity module 1005 may be configured to perform in whole or in part a method 1100, shown in FIG. 11. The method 1100 may be performed in whole or in part by a single computing device, a plurality of electronic devices, and the like. The method 1100 may comprise determining, for each of a plurality of phenotypes, an association score indicative of an association between at least one variant of each gene of a plurality of genes and a phenotype of the plurality of phenotypes at 1110. The association score can indicate a likelihood that the at least one variant is associated with the phenotype. The association score can be determined from GWAS and/or ExWAS data. The association score can comprise one or more of a Z-score, a statistic based on Fisher's method, a rank sum statistic, a p-value, or a combination thereof. The association score can be derived from a regression analysis of GWAS and/or ExWAS data.

The method 1100 may comprise determining, for each gene of the plurality of genes, based on the association scores, a gene-level association score indicative of a representative association between each gene of the plurality of genes and each phenotype of the plurality of phenotypes at 1120. Determining the gene-level association score can comprise determining, for a gene, one or more variants associated with the phenotype, determining, for each of the one or more variants, an association score, and determining, for the gene, based on the association score, the gene-level association score. Determining, for the gene, based on the association score, the gene-level association score can comprise determining the association score with the highest value as gene-level association score or determining an average of the association scores as the gene-level association score.

The method 1100 may comprise generating, based on the gene-level association scores, a gene-phenotype score matrix, wherein the gene-phenotype score matrix comprises, for each gene of the plurality of genes, the gene-level association score for each phenotype of the plurality of phenotypes at 1130.

The method 1100 may comprise receiving a selection of a gene-of-interest at 1140. Receiving a selection of a gene-of-interest can comprise receiving a gene identifier associated with the gene-of-interest.

The method 1100 may comprise determining, based on the selection, in the gene-phenotype score matrix, gene-level association scores of the gene-of-interest at 1150. Determining, based on the selection, in the gene-phenotype score matrix, the gene-of-interest row can comprise determining a row in the gene-phenotype score matrix that comprises the gene identifier associated with the gene-of-interest.

The method 1100 may comprise determining, in the gene-phenotype score matrix, one or more genes associated with gene-level association scores similar to the gene-level association scores of the gene-of-interest at 1160. Determining, in the gene-phenotype score matrix, one or more genes associated with gene-level association scores similar to the gene-level association scores of the gene-of-interest can comprise determining a pairwise similarity between summary association scores of the gene-of-interest and summary association scores of one or more other genes in the gene-phenotype score matrix. Determining, in the gene-phenotype score matrix, one or more genes associated with gene-level association scores similar to the gene-level association scores of the gene-of-interest can comprise generating, based on the gene-phenotype score matrix, a reduced gene-phenotype score matrix, weighting the reduced gene-phenotype score matrix, applying a principal component analysis (PCA) procedure to the weighted reduced gene-phenotype score matrix, and ranking, based on the PCA procedure, relatedness of the one or more genes to the gene-of-interest.

The method 1100 may comprise identifying a gene of the one or more genes as a gene associated with the gene-of-interest at 1170. Identifying a gene of the one or more genes as a gene associated with the gene-of-interest can comprise identifying, from the one or more genes, based on the ranked relatedness, the plurality of genes associated with the gene-of-interest.

The method 1100 may further comprise generating a variant-phenotype association data structure that comprises, for each gene of the plurality of genes, the at least one variant, and the association score of the at least one variant.

The method 1100 may further comprise filtering the variants. Filtering the variants can comprise one or more of: excluding one or more variants that do not map to a protein coding gene, excluding one or more variants that map to an intergenic regions, excluding one or more variants with less than a minimum cell count, or excluding one or more variants associated with a linkage disequilibrium (LD) exceeding a threshold.

The method 1100 may further comprise generating a gene-phenotype score matrix data structure. Generating the gene-phenotype score matrix data structure can comprise generating a logical table, wherein the logical table comprises: a plurality of logical rows, each said logical row including a gene identifier to identify each said logical row, each said logical row corresponding to a record of information, a plurality of logical columns intersecting said plurality of logical rows to define a plurality of logical cells, each said logical column including a phenotype identifier to identify each said logical column, and wherein each of the plurality of logical cells comprises a summary association score.

The gene associated with the gene of interest can be associated with one or more biological pathways. The one or more biological pathways can be signaling pathways, genetic pathways, and/or metabolic pathways. The expression of the gene associated with the gene of interest can be altered.

The method 1100 may further comprise determining a function of the gene associated with the gene of interest and conducting an experiment to assess whether the gene of interest is associated with the function.

The method 1100 may further comprise determining that the gene associated with the gene of interest is a molecular target of a therapeutic agent and conducting an experiment to assess whether the therapeutic agent is associated with a condition related to the gene of interest.

The gene of interest can comprise a knockout target in an organism, and the method 1100 may further comprise determining, that the knockout target does not exist in the first organism, determining that a homolog of the gene associated with the gene of interest exists in the first organism, and utilizing the homolog as the knockout target.

The method 1100 may further comprise determining that modulation of the gene of interest by a therapeutic agent is associated with a negative effect and conducting an experiment to assess whether modulation of the gene associated with the gene of interest by the therapeutic agent is associated with the negative effect.

The method 1100 may further comprise generating, based on the gene of interest and the gene associated with the gene of interest, a gene set and performing, based on the gene set, an enrichment analysis to analyze gene expression data.

The method 1100 may further comprise determining that the gene associated with the gene of interest is associated with a phenotype and conducting an experiment to assess whether the gene of interest is associated with the phenotype.

The method 1100 may further comprise determining a plurality of variants of the gene of interest and the gene associated with the gene of interest and conducting, based on the plurality of variants, an experiment to assess efficacy of a therapeutic agent.

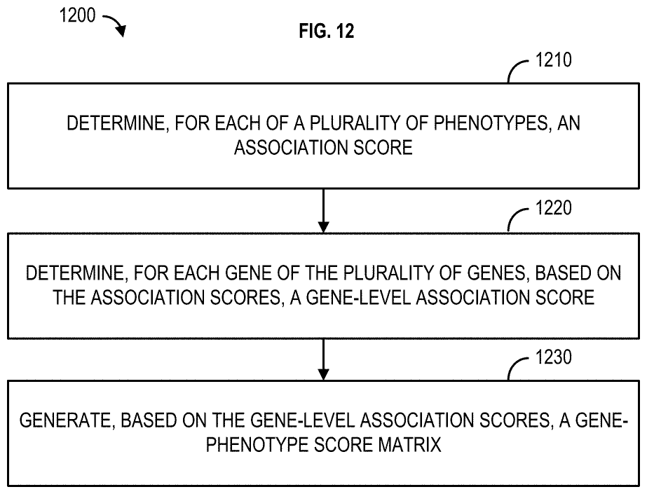

In an embodiment, the similarity module 1005 may be configured to perform in whole or in part a method 1200, shown in FIG. 12. The method 1200 may be performed in whole or in part by a single computing device, a plurality of electronic devices, and the like. The method 1200 may comprise determining, for each of a plurality of phenotypes, an association score indicative of an association between at least one variant of each gene of a plurality of genes and a phenotype of the plurality of phenotypes at 1210. The association score can indicate a likelihood that the at least one variant is associated with the phenotype. The association score can be determined from GWAS and/or ExWAS data. The association score can comprise one or more of a Z-score, a statistic based on Fisher's method, a rank sum statistic, a p-value, or a combination thereof. The association score can be derived from a regression analysis of GWAS and/or ExWAS data.

The method 1200 may comprise determining, for each gene of the plurality of genes, based on the association scores, a gene-level association score indicative of a representative association between each gene of the plurality of genes and each phenotype of the plurality of phenotypes at 1220. Determining the gene-level association score can comprise determining, for a gene, one or more variants associated with the phenotype, determining, for each of the one or more variants, an association score, and determining, for the gene, based on the association score, the gene-level association score. Determining, for the gene, based on the association score, the gene-level association score can comprise determining the association score with the highest value as gene-level association score or determining an average of the association scores as the gene-level association score.

The method 1200 may comprise generating, based on the gene-level association scores, a gene-phenotype score matrix, wherein the gene-phenotype score matrix comprises, for each gene of the plurality of genes, the gene-level association score for each phenotype of the plurality of phenotypes at 1230.

The method 1200 may further comprise generating a variant-phenotype association data structure that comprises, for each gene of the plurality of genes, the at least one variant, and the association score of the at least one variant.

The method 1200 may further comprise filtering the variants. Filtering the variants can comprise one or more of excluding one or more variants that do not map to a protein coding gene, excluding one or more variants that map to an intergenic regions, excluding one or more variants with less than a minimum cell count, or excluding one or more variants associated with a linkage disequilibrium (LD) exceeding a threshold.

The method 1200 may further comprise generating a gene-phenotype score matrix data structure. Generating the gene-phenotype score matrix data structure can comprise generating a logical table, wherein the logical table comprises: a plurality of logical rows, each said logical row including a gene identifier to identify each said logical row, each said logical row corresponding to a record of information, a plurality of logical columns intersecting said plurality of logical rows to define a plurality of logical cells, each said logical column including a phenotype identifier to identify each said logical column, and wherein each of the plurality of logical cells comprises a summary association score.

The method 1200 may further comprise receiving a selection of a gene-of-interest, determining, based on the selection, in the gene-phenotype score matrix, gene-level association scores of the gene-of-interest, determining, in the gene-phenotype score matrix, one or more genes associated with gene-level association scores similar to the gene-level association scores of the gene-of-interest, and identifying a gene of the one or more genes as a gene associated with the gene-of-interest. Receiving the selection of the gene-of-interest can comprise receiving a gene identifier associated with the gene-of-interest. Determining, based on the selection, in the gene-phenotype score matrix, the gene-of-interest row can comprise determining a row in the gene-phenotype score matrix that comprises the gene identifier associated with the gene-of-interest. Determining, in the gene-phenotype score matrix, one or more genes associated with gene-level association scores similar to the gene-level association scores of the gene-of-interest can comprise determining a pairwise similarity between summary association scores of the gene-of-interest and summary association scores of one or more other genes in the gene-phenotype score matrix. Determining, in the gene-phenotype score matrix, one or more genes associated with gene-level association scores similar to the gene-level association scores of the gene-of-interest can comprise generating, based on the gene-phenotype score matrix, a reduced gene-phenotype score matrix, weighting the reduced gene-phenotype score matrix, applying a principal component analysis (PCA) procedure to the weighted reduced gene-phenotype score matrix, and ranking, based on the PCA procedure, relatedness of the one or more genes to the gene-of-interest. Identifying a gene of the one or more genes as a gene associated with the gene-of-interest can comprise identifying, from the one or more genes, based on the ranked relatedness, the plurality of genes associated with the gene-of-interest.

The gene associated with the gene of interest can be associated with one or more biological pathways. The one or more biological pathways can be signaling pathways, genetic pathways, and/or metabolic pathways. The expression of the gene associated with the gene of interest can be altered.

The method 1200 may further comprise determining a function of the gene associated with the gene of interest and conducting an experiment to assess whether the gene of interest is associated with the function.

The method 1200 may further comprise determining that the gene associated with the gene of interest is a molecular target of a therapeutic agent and conducting an experiment to assess whether the therapeutic agent is associated with a condition related to the gene of interest.

The gene of interest can comprise a knockout target in an organism, and the method 1200 may further comprise determining, that the knockout target does not exist in the first organism, determining that a homolog of the gene associated with the gene of interest exists in the first organism, and utilizing the homolog as the knockout target.

The method 1200 may further comprise determining that modulation of the gene of interest by a therapeutic agent is associated with a negative effect and conducting an experiment to assess whether modulation of the gene associated with the gene of interest by the therapeutic agent is associated with the negative effect.

The method 1200 may further comprise generating, based on the gene of interest and the gene associated with the gene of interest, a gene set and performing, based on the gene set, an enrichment analysis to analyze gene expression data.

The method 1200 may further comprise determining that the gene associated with the gene of interest is associated with a phenotype and conducting an experiment to assess whether the gene of interest is associated with the phenotype.

The method 1200 may further comprise determining a plurality of variants of the gene of interest and the gene associated with the gene of interest and conducting, based on the plurality of variants, an experiment to assess efficacy of a therapeutic agent.

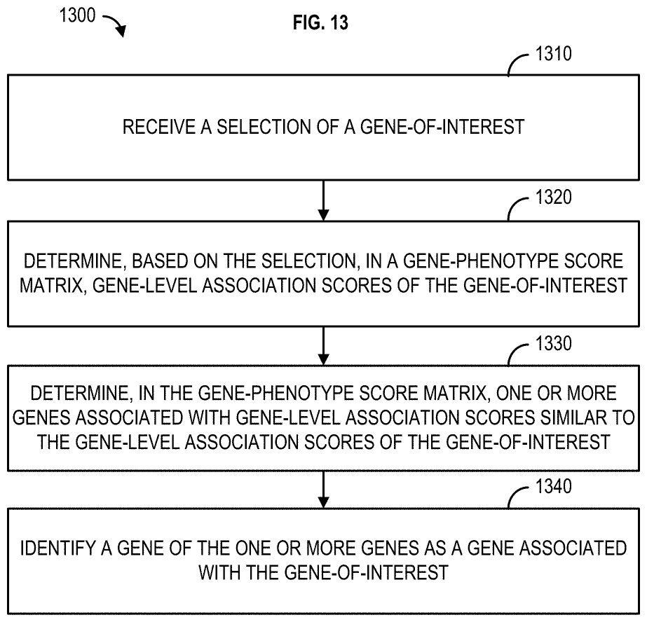

In an embodiment, the similarity module 1005 may be configured to perform in whole or in part a method 1300, shown in FIG. 13. The method 1300 may be performed in whole or in part by a single computing device, a plurality of electronic devices, and the like. The method 1300 may comprise receiving a selection of a gene-of-interest at 1310. Receiving a selection of a gene-of-interest can comprise receiving a gene identifier associated with the gene-of-interest.

The method 1300 may comprise determining, based on the selection, in a gene-phenotype score matrix, gene-level association scores of the gene-of-interest, wherein the gene-phenotype score matrix comprises, for each gene of a plurality of genes, a gene-level association score for each phenotype of a plurality of phenotypes at 1320. Determining, based on the selection, in the gene-phenotype score matrix, the gene-of-interest row can comprise determining a row in the gene-phenotype score matrix that comprises the gene identifier associated with the gene-of-interest.

The method 1300 may comprise determining, in the gene-phenotype score matrix, one or more genes associated with gene-level association scores similar to the gene-level association scores of the gene-of-interest at 1330. Determining, in the gene-phenotype score matrix, one or more genes associated with gene-level association scores similar to the gene-level association scores of the gene-of-interest can comprise determining a pairwise similarity between summary association scores of the gene-of-interest and summary association scores of one or more other genes in the gene-phenotype score matrix. Determining, in the gene-phenotype score matrix, one or more genes associated with gene-level association scores similar to the gene-level association scores of the gene-of-interest can comprise generating, based on the gene-phenotype score matrix, a reduced gene-phenotype score matrix, weighting the reduced gene-phenotype score matrix, applying a principal component analysis (PCA) procedure to the weighted reduced gene-phenotype score matrix, and ranking, based on the PCA procedure, relatedness of the one or more genes to the gene-of-interest.

The method 1300 may comprise identifying a gene of the one or more genes as a gene associated with the gene-of-interest at 1340. Identifying a gene of the one or more genes as a gene associated with the gene-of-interest comprises identifying, from the one or more genes, based on the ranked relatedness, the plurality of genes associated with the gene-of-interest.

The method 1300 may further comprise determining, for each of a plurality of phenotypes, an association score indicative of an association between at least one variant of each gene of a plurality of genes and a phenotype of the plurality of phenotypes, determining, for each gene of the plurality of genes, based on the association scores, a gene-level association score indicative of a representative association between each gene of the plurality of genes and each phenotype of the plurality of phenotypes, generating, based on the gene-level association scores, a gene-phenotype score matrix, wherein the gene-phenotype score matrix comprises, for each gene of the plurality of genes, the gene-level association score for each phenotype of the plurality of phenotypes. The association score can indicate a likelihood that the at least one variant is associated with the phenotype. The association score can be determined from GWAS and/or ExWAS data. The association score can comprise one or more of a Z-score, a statistic based on Fisher's method, a rank sum statistic, a p-value, or a combination thereof. The association score can be derived from a regression analysis of GWAS and/or ExWAS data. Determining the gene-level association score can comprise determining, for a gene, one or more variants associated with the phenotype, determining, for each of the one or more variants, an association score, and determining, for the gene, based on the association score, the gene-level association score. Determining, for the gene, based on the association score, the gene-level association score can comprise determining the association score with the highest value as gene-level association score or determining an average of the association scores as the gene-level association score.

The method 1300 may further comprise generating a variant-phenotype association data structure that comprises, for each gene of the plurality of genes, the at least one variant, and the association score of the at least one variant.

The method 1300 may further comprise filtering the variants. Filtering the variants can comprise one or more of excluding one or more variants that do not map to a protein coding gene, excluding one or more variants that map to an intergenic regions, excluding one or more variants with less than a minimum cell count, or excluding one or more variants associated with a linkage disequilibrium (LD) exceeding a threshold.

The method 1300 may further comprise generating a gene-phenotype score matrix data structure. Generating the gene-phenotype score matrix data structure can comprise generating a logical table, wherein the logical table comprises: a plurality of logical rows, each said logical row including a gene identifier to identify each said logical row, each said logical row corresponding to a record of information, a plurality of logical columns intersecting said plurality of logical rows to define a plurality of logical cells, each said logical column including a phenotype identifier to identify each said logical column, and wherein each of the plurality of logical cells comprises a summary association score.

The gene associated with the gene of interest can be associated with one or more biological pathways. The one or more biological pathways can be signaling pathways, genetic pathways, and/or metabolic pathways. The expression of the gene associated with the gene of interest can be altered.

The method 1300 may further comprise determining a function of the gene associated with the gene of interest and conducting an experiment to assess whether the gene of interest is associated with the function.

The method 1300 may further comprise determining that the gene associated with the gene of interest is a molecular target of a therapeutic agent and conducting an experiment to assess whether the therapeutic agent is associated with a condition related to the gene of interest.

The gene of interest can comprise a knockout target in an organism, and the method 1300 may further comprise determining, that the knockout target does not exist in the first organism, determining that a homolog of the gene associated with the gene of interest exists in the first organism, and utilizing the homolog as the knockout target.

The method 1300 may further comprise determining that modulation of the gene of interest by a therapeutic agent is associated with a negative effect and conducting an experiment to assess whether modulation of the gene associated with the gene of interest by the therapeutic agent is associated with the negative effect.

The method 1300 may further comprise generating, based on the gene of interest and the gene associated with the gene of interest, a gene set and performing, based on the gene set, an enrichment analysis to analyze gene expression data.

The method 1300 may further comprise determining that the gene associated with the gene of interest is associated with a phenotype and conducting an experiment to assess whether the gene of interest is associated with the phenotype.

The method 1300 may further comprise determining a plurality of variants of the gene of interest and the gene associated with the gene of interest and conducting, based on the plurality of variants, an experiment to assess efficacy of a therapeutic agent.

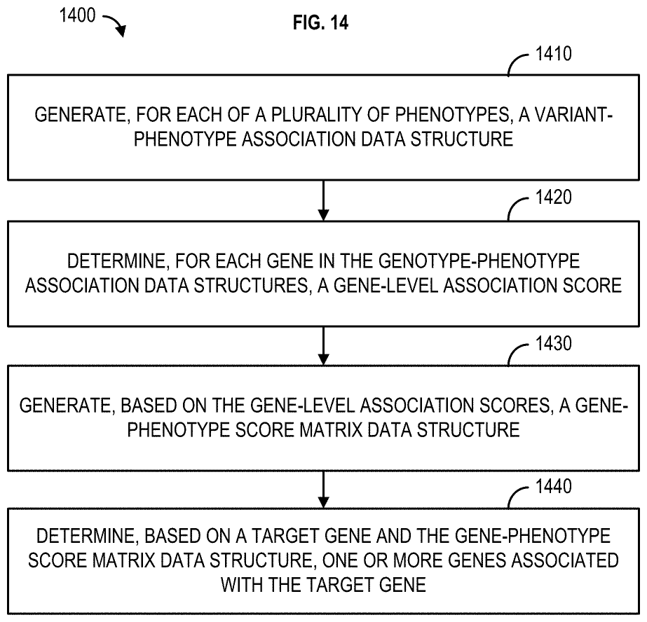

In an embodiment, the similarity module 1005 may be configured to perform in whole or in part a method 1400, shown in FIG. 14. The method 1400 may be performed in whole or in part by a single computing device, a plurality of electronic devices, and the like. The method 1400 may comprise generating, for each of a plurality of phenotypes, a variant-phenotype association data structure at 1410. The variant-phenotype association data structure can comprise, for each gene of a plurality of genes, at least one variant and an association score of the at least one variant. The association score can indicate a likelihood that the at least one variant is associated with the phenotype. The association score can be determined from GWAS and/or ExWAS data. The association score can comprise one or more of a Z-score, a statistic based on Fisher's method, a rank sum statistic, a p-value, or a combination thereof. The association score can be derived from a regression analysis of GWAS and/or ExWAS data.

The method 1400 may comprise determining, for each gene in the genotype-phenotype association data structures, a gene-level association score at 1420. Determining the gene-level association score can comprise determining, for a gene, one or more variants associated with the phenotype, determining, for each of the one or more variants, an association score, and determining, based on the association score, the gene-level association score. Determining, based on the association score, the gene-level association score can comprise determining the association score with the highest value as the gene-level association score, or determining an average of the association scores as the gene-level association score.

The method 1400 may comprise generating, based on the gene-level association scores, a gene-phenotype score matrix data structure at 1430. The gene-phenotype score matrix data structure can comprise, for each gene of a plurality of genes, a gene-level association score for each phenotype of the plurality of phenotypes. Generating the gene-phenotype score matrix data structure can comprise generating a logical table, wherein the logical table can comprise a plurality of logical rows, each said logical row including a gene identifier to identify each said logical row, each said logical row corresponding to a record of information, a plurality of logical columns intersecting said plurality of logical rows to define a plurality of logical cells, each said logical column including a phenotype identifier to identify each said logical column, and wherein each of the plurality of logical cells comprises a summary association score.

The method 1400 may comprise determining, based on a target gene and the gene-phenotype score matrix data structure, one or more genes associated with the target gene at 1440. Determining, based on the target gene and the gene-phenotype score matrix data structure, one or more genes associated with the target gene can comprise generating, based on the gene-phenotype score matrix data structure, a reduced gene-phenotype score matrix data structure, weighting the reduced gene-phenotype score matrix data structure, applying a principal component analysis (PCA) procedure to the weighted reduced gene-phenotype score matrix data structure, ranking, based on the PCA procedure, relatedness of a plurality of genes to the target gene, and identifying, from the plurality of genes, based on the relatedness, the one or more genes associated with the target gene. Determining, based on the target gene and the gene-phenotype score matrix data structure, one or more genes associated with the target gene can comprise determining a pairwise similarity between summary association scores of the target gene and summary association scores of one or more other genes in the gene-phenotype score matrix data structure.

The method 1400 may further comprise filtering the variant-phenotype association data structure. Filtering the variant-phenotype association data structure comprises one or more of excluding one or more variants that do not map to a protein coding gene, excluding one or more variants that map to an intergenic regions, excluding one or more variants with less than a minimum cell count, or excluding one or more variants associated with a linkage disequilibrium (LD) exceeding a threshold.

The one or more genes associated with the target gene are associated with one or more biological pathways. The one or more biological pathways are signaling pathways, genetic pathways, and/or metabolic pathways. Expression of the one or more genes associated with the target gene can be altered.

The method 1400 may further comprise determining a function of the one or more genes associated with the target gene and conducting an experiment to assess whether the target gene is associated with the function.

The method 1400 may further comprise determining that the one or more genes associated with the target gene is a molecular target of a therapeutic agent and conducting an experiment to assess whether the therapeutic agent is associated with a condition related to the target gene.

The target gene can comprise a knockout target in an organism, and the method 1400 may further comprise determining, that the knockout target does not exist in the first organism, determining that a homolog of the one or more genes associated with the target gene exists in the first organism, and utilizing the homolog as the knockout target.

The method 1400 may further comprise determining that modulation of the target gene by a therapeutic agent is associated with a negative effect and conducting an experiment to assess whether modulation of the one or more genes associated with the target gene by the therapeutic agent is associated with the negative effect.

The method 1400 may further comprise generating, based on the target gene and the one or more genes associated with the target gene, a gene set and performing, based on the gene set, an enrichment analysis to analyze gene expression data.

The method 1400 may further comprise determining that the one or more genes associated with the target gene is associated with a phenotype and conducting an experiment to assess whether the target gene is associated with the phenotype.

The method 1400 may further comprise determining a plurality of variants of the target gene and the one or more genes associated with the target gene and conducting, based on the plurality of variants, an experiment to assess efficacy of a therapeutic agent.

Those skilled in the art will recognize, or be able to ascertain using no more than routine experimentation, many equivalents to the specific embodiments of the method and compositions described herein. Such equivalents are intended to be encompassed by the following claims.

We claim:

1. A method comprising:
determining, for each of a plurality of phenotypes, an association score indicative of an association between at least one variant of each gene of a plurality of genes and a phenotype of the plurality of phenotypes;
determining, for each gene of the plurality of genes, based on the association scores, a gene-level association score indicative of a representative association between each gene of the plurality of genes and each phenotype of the plurality of phenotypes;
generating, based on the gene-level association scores, a gene-phenotype score matrix, wherein the gene-phenotype score matrix comprises, for each gene of the plurality of genes, the gene-level association score for each phenotype of the plurality of phenotypes;
receiving a selection of a gene-of-interest;
determining, based on the selection, in the gene-phenotype score matrix, gene-level association scores of the gene-of-interest;
determining, in the gene-phenotype score matrix, one or more genes associated with gene-level association scores similar to the gene-level association scores of the gene-of-interest; and
identifying a gene of the one or more genes as a gene associated with the gene-of-interest.

2. The method of claim 1, wherein the association score indicates a likelihood that the at least one variant is associated with the phenotype, wherein the association score comprises one or more of a Z-score, a statistic based on Fisher's method, a rank sum statistic, a p-value, or a combination thereof.

3. The method of claim 1, further comprising generating a variant-phenotype association data structure that comprises, for each gene of the plurality of genes, the at least one variant, and the association score of the at least one variant.

4. The method of claim 1, further comprising filtering the variants, wherein filtering the variants comprises one or more of:
excluding one or more variants that do not map to a protein coding gene;
excluding one or more variants that map to an intergenic regions;
excluding one or more variants with less than a minimum cell count; or
excluding one or more variants associated with a linkage disequilibrium (LD) exceeding a threshold.

5. The method of claim 1, wherein determining the gene-level association score comprises:
determining, for a gene, one or more variants associated with the phenotype;
determining, for each of the one or more variants, an association score;
determining the association score with the highest value as gene-level association score; or
determining an average of the association scores as the gene-level association score.

6. The method of claim 1, further comprising generating a gene-phenotype score matrix data structure, wherein generating the gene-phenotype score matrix data structure comprises:
generating a logical table, wherein the logical table comprises:
a plurality of logical rows, each said logical row including a gene identifier to identify each said logical row, each said logical row corresponding to a record of information;
a plurality of logical columns intersecting said plurality of logical rows to define a plurality of logical cells, each said logical column including a phenotype identifier to identify each said logical column; and
wherein each of the plurality of logical cells comprises a summary association score.

7. The method of claim 1, wherein receiving a selection of a gene-of-interest comprises receiving a gene identifier associated with the gene-of-interest and wherein determining, based on the selection, in the gene-phenotype score matrix, the gene-of-interest row comprises determining a row in the gene-phenotype score matrix that comprises the gene identifier associated with the gene-of-interest.

8. The method of claim 1, wherein determining, in the gene-phenotype score matrix, one or more genes associated with gene-level association scores similar to the gene-level association scores of the gene-of-interest comprises determining a pairwise similarity between summary association scores of the gene-of-interest and summary association scores of one or more other genes in the gene-phenotype score matrix.

9. The method of claim 1, wherein determining, in the gene-phenotype score matrix, one or more genes associated with gene-level association scores similar to the gene-level association scores of the gene-of-interest comprises:
generating, based on the gene-phenotype score matrix, a reduced gene-phenotype score matrix;
weighting the reduced gene-phenotype score matrix;
applying a principal component analysis (PCA) procedure to the weighted reduced gene-phenotype score matrix;
ranking, based on the PCA procedure, relatedness of the one or more genes to the gene-of-interest; and
wherein identifying a gene of the one or more genes as a gene associated with the gene-of-interest comprises identifying, from the one or more genes, based on the ranked relatedness, the plurality of genes associated with the gene-of-interest.

10. The method of claim 1, wherein the gene associated with the gene of interest is associated with one or more biological pathways, wherein the one or more biological pathways are signaling pathways, genetic pathways, and/or metabolic pathways.

11. The method of claim 1, further comprising:
determining a function of the gene associated with the gene of interest; and
conducting an experiment to assess whether the gene of interest is associated with the function.

12. The method of claim 1, further comprising:
determining that the gene associated with the gene of interest is a molecular target of a therapeutic agent; and
conducting an experiment to assess whether the therapeutic agent is associated with a condition related to the gene of interest.

13. The method of claim 1, wherein the gene of interest comprises a knockout target in an organism, wherein the method further comprises:
determining, that the knockout target does not exist in the first organism;
determining that a homolog of the gene associated with the gene of interest exists in the first organism; and
utilizing the homolog as the knockout target.

14. The method of claim 1, further comprising:
determining that modulation of the gene of interest by a therapeutic agent is associated with a negative effect; and
conducting an experiment to assess whether modulation of the gene associated with the gene of interest by the therapeutic agent is associated with the negative effect.

15. The method of claim 1, further comprising:
generating, based on the gene of interest and the gene associated with the gene of interest, a gene set; and
performing, based on the gene set, an enrichment analysis to analyze gene expression data.

16. The method of claim 1, further comprising:
determining that the gene associated with the gene of interest is associated with a phenotype; and
conducting an experiment to assess whether the gene of interest is associated with the phenotype.

17. The method of claim 1, further comprising:
determining a plurality of variants of the gene of interest and the gene associated with the gene of interest; and
conducting, based on the plurality of variants, an experiment to assess efficacy of a therapeutic agent.

18. The method of claim 1, further comprising:
administering a therapeutic agent to a subject,
wherein the subject has been determined to have a phenotype associated with the gene-of-interest,
wherein the therapeutic agent alters expression of the gene associated with the gene-of-interest, and
wherein the altered expression of the gene associated with the gene-of-interest provides a therapeutic effect to the subject.

19. The method of claim 18, wherein the altered expression is an increase in expression of the gene associated with the gene-of-interest, wherein an increase in expression provides a therapeutic effect.

20. The method of claim 18, wherein the altered expression is a decrease in expression of the gene associated with the gene-of-interest, wherein a decrease in expression provides a therapeutic effect.

21. A method comprising:
determining, for each of a plurality of phenotypes, an association score indicative of an association between at least one variant of each gene of a plurality of genes and a phenotype of the plurality of phenotypes;
determining, for each gene of the plurality of genes, based on the association scores, a gene-level association score indicative of a representative association between each gene of the plurality of genes and each phenotype of the plurality of phenotypes; and
generating, based on the gene-level association scores, a gene-phenotype score matrix, wherein the gene-phenotype score matrix comprises, for each gene of the plurality of genes, the gene-level association score for each phenotype of the plurality of phenotypes.

22. A method comprising:
receiving a selection of a gene-of-interest;
determining, based on the selection, in a gene-phenotype score matrix, gene-level association scores of the gene-of-interest, wherein the gene-phenotype score matrix comprises, for each gene of a plurality of genes, a gene-level association score for each phenotype of a plurality of phenotypes;
determining, in the gene-phenotype score matrix, one or more genes associated with gene-level association scores similar to the gene-level association scores of the gene-of-interest; and
identifying a gene of the one or more genes as a gene associated with the gene-of-interest.
